(19) United States
(12) Patent Application Publication
(10) Pub. No.: US 2020/0219595 A1
(43) Pub. Date: Jul. 9, 2020

(54) MEDICAL INFORMATION DISPLAY APPARATUS AND MEDICAL INFORMATION DISPLAY SYSTEM

(71) Applicant: Canon Medical Systems Corporation, Otawara-shi (JP)

(72) Inventor: Masashi YOSHIDA, Nasushiobara (JP)

(73) Assignee: Canon Medical Systems Corporation, Otawara-shi (JP)

(21) Appl. No.: 16/718,476

(22) Filed: Dec. 18, 2019

(30) Foreign Application Priority Data
Dec. 19, 2018 (JP) 2018-237442
Dec. 16, 2019 (JP) 2019-226395

(51) Int. Cl. G16H 10/60; G16H 50/70; G16H 40/63; G16H 30/20; G16H 15/00

(52) U.S. Cl. CPC G16H 10/60; G16H 50/70; G16H 15/00; G16H 30/20; G16H 40/63

(57) ABSTRACT

According to one embodiment, a medical information display apparatus includes processing circuitry. The processing circuitry retrieves, based on subject patient information on a subject patient, similar-case patient information on a similar-case patient having a case similar to a case of the subject patient. The processing circuitry detects difference information of a difference between the subject patient information and the similar-case patient information which is equal to or greater than a first threshold. The processing circuitry causes the subject patient information and the similar-case patient information to be displayed and highlight the difference information.

### CROSS-REFERENCE TO RELATED APPLICATIONS

This application is based upon and claims the benefit of priority from the prior Japanese Patent Application No. 2018-237442, filed Dec. 19, 2018, and No. 2019-226395, filed Dec. 16, 2019, the entire contents of all of which are incorporated herein by reference.

### FIELD

Embodiments described herein relate generally to a medical information display apparatus and a medical information display system.

### BACKGROUND

In recent years, an information integrated display viewer, which displays a list of medical information distributed among a plurality of systems, has been introduced into hospitals. Use of the information integrated display viewer enables retrieval of a similar case or a patient with a similar case to the case subject patient, and enables display with the timelines aligned.

However, simply aligning the timelines and comparing the subject patient and the similar-case patient or their cases may result in oversight when there is a minor difference therebetween.

### BRIEF DESCRIPTION OF THE DRAWINGS

FIG. 1 is a block diagram showing a configuration example of a medical information display apparatus according to the present embodiment.

FIG. 2 is a flowchart of an operation of the medical information display apparatus according to the present embodiment.

FIG. 3 is a diagram showing a first example of time scale alignment by an alignment function.

FIG. 4 is a diagram showing a second example of time scale alignment by the alignment function.

FIG. 5 is a flowchart showing details of highlighting processing of the medical information display apparatus according to the present embodiment.

FIG. 6 is a diagram showing a first example of highlighting according to the present embodiment.

FIG. 7 is a diagram showing a second example of highlighting according to the present embodiment.

FIG. 8 is a diagram showing a third example of highlighting according to the present embodiment.

FIG. 9 is a diagram showing a fourth example of highlighting according to the present embodiment.

### DETAILED DESCRIPTION

In general, according to one embodiment, a medical information display apparatus includes processing circuitry. The processing circuitry retrieves similar-case patient information based on subject patient information, the subject patient information including time-series treatment information on a subject patient, the similar-case patient information including time-series treatment information on a similar-case patient which has a case similar to a case of the subject patient. The processing circuitry detects, as difference information, information of a difference between the subject patient information and the similar-case patient information which is equal to or greater than a first threshold. The processing circuitry causes the subject patient information and the similar-case patient information to be displayed and highlight the difference information.

Hereinafter, a medical information display apparatus and medical information display system according to the present embodiment will be described with reference to the accompanying drawings. In the following embodiment, elements assigned the same reference numerals perform the same operations, and redundant descriptions will be omitted as appropriate.

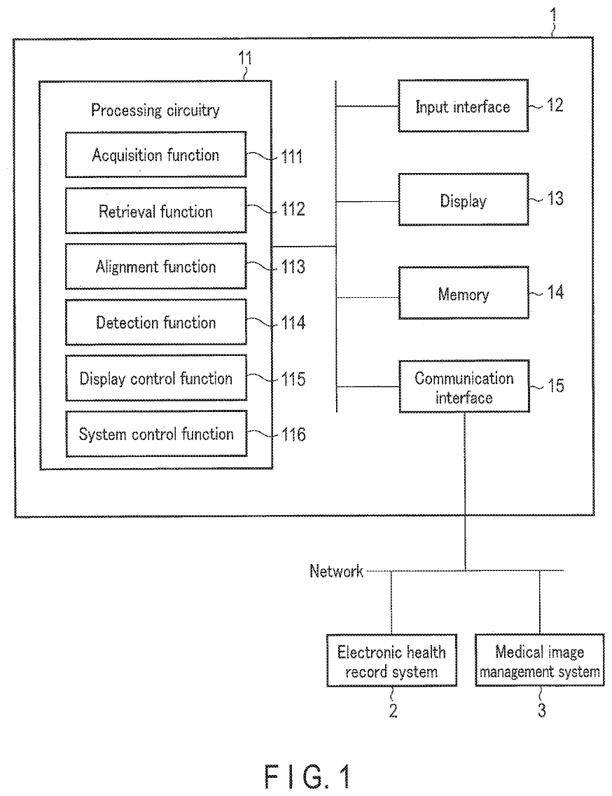

FIG. 1 is a block diagram showing a medical information display apparatus 1 according to the present embodiment. The medical information display apparatus 1 shown in FIG. 1 is connected to a medical information management system, such as an electronic health record system (or an electronic medical record system) 2, or a medical image management system (picture archiving and communication system (PACS)) 3, through a network, and exchanges various data with the system. The network may be either wired or wireless. As long as security is ensured, the connection may be made through a network other than the intra-hospital network. For example, the connection may be made through a public communication line, such as the Internet, via a virtual private network (VPN) or the like.

The medical information display apparatus 1 includes processing circuitry 11, an input interface 12, a display 13, a memory 14, and a communication interface 15. The processing circuitry 11, the input interface 12, the display 13, the memory 14, and the communication interface 15 are communicatively connected to one another via, for example, a bus.

The medical information display apparatus 1 may be a computer installed in a medical image diagnosis apparatus in which a medical imaging apparatus is installed, a computer communicatively connected to the medical image diagnosis apparatus via a cable, a network, etc., or a computer, such as a workstation, independent from the medical image diagnosis apparatus.

The electronic health record system 2 is a system that stores electronic health record data including patient identification information and treatment information associated with the patient identification information, etc., and manages the stored electronic health record data. The treatment information includes information in an electronic health record, such as information on findings, disease name information, vital sign information, test stage information, a clinical pathway, and information on details of treatment. The patient identification information includes a patient ID, a patient's name, sex, age, etc. The clinical pathway refers to a time-series standard treatment plan.

The medical image management system 3 is a system that stores medical image data and manages the stored medical image data. The medical image management system 3 stores and manages medical image data converted in accordance with, for example, the Digital Imaging and Communications in Medicine (DICOM) standard.

Hereinafter, the medical information display apparatus 1 will be described in more detail. The processing circuitry 11 is, for example, a processor and includes an acquisition function 111, a retrieval function 112, an alignment function 113, a detection function 114, a display control function 115, and a system control function 116. The functions 111 to 116 are not necessarily implemented by a single processing circuit. Processing circuitry may be configured by combining a plurality of independent processors that execute respective programs to implement the respective functions 111-116.

Each of the functions 111 to 116 may be stored as a program in, for example, the memory 14, and the processing circuitry 11 may execute the stored program, thereby implementing a function corresponding to the executed program.

By executing the acquisition function 111, the processing circuitry 11 acquires subject patient information including patient identification information and time-series treatment information on a subject patient which is to be processed.

By executing the retrieval function 112, the processing circuitry 11 retrieves similar-case patient information including time-series treatment information on a similar-case patient of a case similar to the case of the subject patient on the basis of the subject patient information. The similar-case patient information may include time-series treatment information on past treatments of the subject patient, such as the treatment before the last and the last treatment of the subject patient.

By executing the alignment function 113, the processing circuitry 11 performs alignment so that, when the subject patient information and the similar-case patient information are displayed, the time scales of the timelines are aligned at least in part.

By executing the detection function 114, the processing circuitry 11 detects, as difference information, information of a difference between the subject patient information and the similar-case patient information which is equal to or greater than a threshold, and detects improper information in the subject patient information and the similar-case patient information. The improper information is, for example, an abnormal value or an outlier.

By executing the display control function 115, the processing circuitry 11 causes the subject patient information and the similar-case patient information to be displayed and highlights at least one of the difference information and the improper information.

By executing the system control function 116, the processing circuitry 11 controls basic operations, such as input/output and communication, of the medical information display apparatus 1. By executing the system control function 116, the processing circuitry 11 receives various requests via the input interface 12. The processing circuitry 11 performs various functions in accordance with various received requests.

The input interface 12 receives various input operations from a user, converts the received input operations into electrical signals, and outputs the electrical signals to the processing circuitry 11. The input interface is connected to an input device, such as a mouse, a keyboard, a trackball, a switch, a button, joystick, a touch-pad, or a touch panel to which an instruction is input by the user touching the operation screen. The input device connected to the input interface may be an input device provided in another computer connected through a network, etc.

The display 13 displays various types of information in accordance with an instruction from the processing circuitry 11. The display may display, for example, a graphical user interface (GUI) for receiving various operations from the user. As the display, any display, such as a cathode ray tube (CRT) display, a liquid crystal display, an organic electroluminescence display (OELD), light-emitting diode (LED) display, or a plasma display, may be used as appropriate. It is possible not to include the display 13 in the medical information display apparatus 1 and to display a GUI on an external display, or cause a GUI to be displayed through a projector or the like.

The memory 14 is a storage device, such as a read-only memory (ROM), a random access memory (RAM), a hard disk drive (HDD), a solid state drive (SSD), or an integrated circuit storage device, which stores various information. The memory 14 may be, for example, a CD-ROM drive, a DVD drive, or a drive which reads and writes various types of information from and in a portable storage medium such as a flash memory. The memory 14 need not necessarily be implemented by a single storage device, but may be implemented by a plurality of storage devices. The memory 14 may be in another computer connected to the medical information display apparatus 1 via a network.

The communication interface 15 stores, for example, a medical information display program according to the present embodiment. The medical information display program may be stored in advance in, for example, the memory 14. Alternatively, the medical information display program may be, for example, stored in a non-transitory storage medium and distributed, and then read from the non-transitory storage medium and installed in the memory 14.

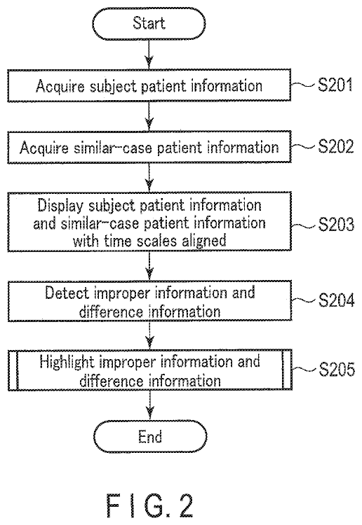

Next, an operation example of the medical information display apparatus 1 according to the present embodiment will be described with reference to the flowchart of FIG. 2.

In step S201, the processing circuitry 11 acquires subject patient information by executing the acquisition function 111. The subject patient information acquired here includes SOAP, anamnesis, a test image, and the like, in addition to the above-described patient identification information and treatment information. SOAP represents Subject (subjective data), Object (objective data), Assessment (an assessment on the subjective data and objective data), and Plan (a treatment policy based on the above three items), and is an analysis method. The subject patient information may be acquired from the electronic health record system 2 via a network. When the subject patient information is a medical image in particular, the information may be acquired from the medical image management system 3.

In step S202, the processing circuitry 11 retrieves similar-case patient information on a similar-case patient through the retrieval function 112. As a method for retrieving similar-case patient information, the processing circuitry 11 uses part of the subject patient information, for example, the age, sex, weight, symptoms, and disease name, as retrieval conditions to select a range of treatment information to be extracted as similar-case patient information, and searches the electronic health record system 2 and the medical image management system 3 in accordance with the retrieval conditions, by executing the retrieval function 112. The processing circuitry 11 determines a primary population of patient identification information that matches the retrieval conditions and the corresponding treatment information, by executing the retrieval function 112. By executing the retrieval function 112, the processing circuitry 11 may determine, from the determined primary population, a secondary population in which the degree of similarity in information of interest (such as symptom or test result) is equal to or greater than a threshold, and then determine, from the secondary population, a patient of the case with the highest degree of similarity as the similar-case patient. The processing circuitry 11 extracts patient identification information on the similar-case patient and treatment information associated with the patient identification information as similar-case patient information, by executing the retrieval function 112.

The past treatment information of the subject patient, treatment information of others, and the like may be made selectable as retrieval conditions. As items of the above-mentioned retrieval condition, “self” and “others” are prepared, for example. Let us assume that a user selects “self” and “others”. In this case, the processing circuitry 11 may determine, by executing the retrieval function 112, a primary population from past treatment information of the subject patient in addition to the treatment information of others. Of course, if the item “self” or “others” is not selected, treatment information of the non-selected item may be excluded from the retrieval objects.

If the secondary population is small, the condition for determining the primary population may be relaxed to increase the number of samples to be included in the primary population. For example, where only treatment information of “20s” is extracted while using the age as a retrieval condition, treatment information of “30s” may be added to the retrieval objects

By executing the retrieval function 112, the processing circuitry 11 may extract not only a patient of a case with the highest degree of similarity to the case of the subject patient, but also other patients as similar-case patients, and cause them to be displayed in the next step S203, or may average treatment information on the extracted similar-case patients to calculate a similar case and cause it to be displayed in the next step S203.

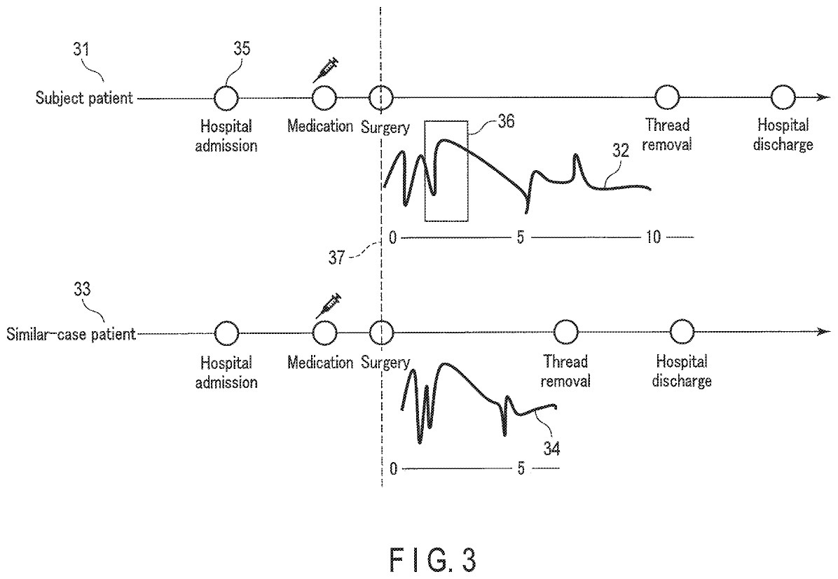

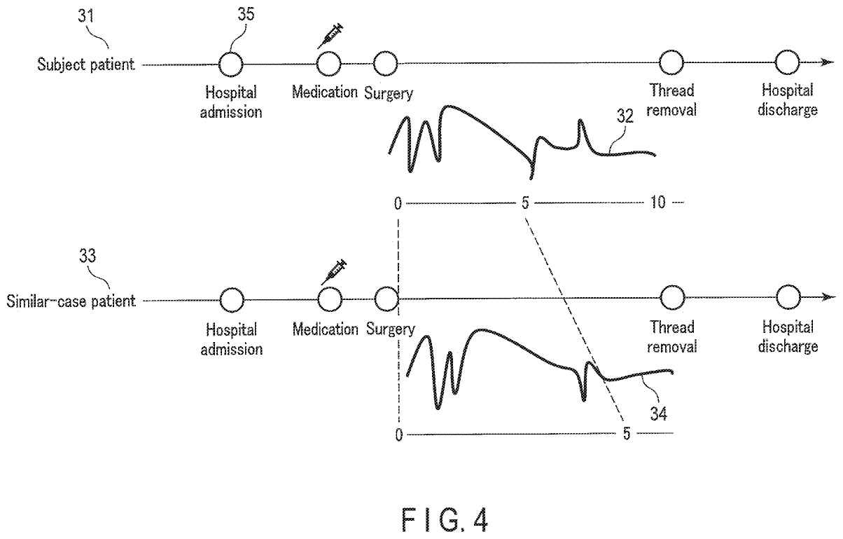

In step S203, the processing circuitry 11 causes the subject patient information and similar-case patient information to be displayed on the GUI with the time scales aligned, by executing the alignment function 113 and the display control function 115. The method for aligning the time scales will be described later with reference to FIGS. 3 and 4.

In step S204, the processing circuitry 11 detects improper information and difference information based on the subject patient information and similar-case patient information by executing the detection function 114. The improper information is information indicating an outlier or abnormal value detected when there is a value that, for example, deviates from a reference value by a threshold or more. The difference information is information of values for which a difference is equal to or greater than a threshold, which is detected when the subject patient information is compared with the similar-case patient information on the aligned time scales with respect to the same item.

For difference information relating to a difference between continuous values, difference information may be detected by performing pattern matching after aligning the time scales by extension and contraction, or may be detected by performing pattern matching before alignment of the time scales, i.e., before step S203.

In step S205, the processing circuitry highlights, by executing the display control function 115, improper information and difference information on the GUI on which the subject patient information and similar-case patient information are displayed.

Next, a first example of time scale alignment by the alignment function 113 will be described with reference to FIG. 3.

The upper part of FIG. 3 is a graph showing treatment information 31 of a subject patient in association with test information 32 (here, systolic blood pressure) of the subject patient along the same timeline. The lower part in FIG. 3 is a graph showing treatment information 33 of a similar-case patient in association with test information 34 of the similar-case patient along the same timeline.

Treatment information 31 and treatment information 33 are acquired from treatment information stored in the electronic health record system 2, and events 35 of the clinical pathway are shown for the sake of explanation. The events 35 of the clinical pathway included in treatment information 31 and treatment information 33 are assumed to be “hospital admission”, “medication”, “surgery”, “thread removal”, “hospital discharge” herein. As test information 32 and test information 34, the systolic blood pressures corresponding to the days from “surgery” to “thread removal” are shown herein.

In the first example of alignment, the time scales are aligned based on information of interest 36 designated by the user. Specifically, given information of interest 36 in the test information 32 of the subject patient is designated. The processing circuitry 11 sets, by executing the alignment function 113, an event immediately before the information of interest 36 as a reference event 37 with reference to the information of interest 36 (portion of interest in the example of FIG. 3) designated by the user or the information of interest 36 automatically designated, such as the maximum value in the graph. Thereafter, the processing circuitry 11 aligns, by executing the alignment function 113, the time scales of the subject patient and the similar-case patient with each other with reference to the reference event 37.

In the example of FIG. 3, the event 35 immediately before the information of interest 36 is “surgery”; therefore, the processing circuitry 11 aligns the time scales while referring to “surgery” as the reference event 37 through the alignment function 113. Consequently, when the date of the reference event 37 “surgery” is regarded as “0 (zero)”, it can be understood that it takes approximately six days from “surgery” to “thread removal” according to the treatment information 33 of the similar-case patient, whereas it takes approximately ten days from “surgery” to “thread removal” according to the treatment information 31 of the subject patient and, for example, a comparison as to the degree of recovery after the surgery can be made.

When the treatment information 33 of the similar-case patient is past treatment information of the subject patient, a comparison to detect that the present recovery process takes more time than the previous recovery process, which means that the present recovery is slow, can be made. By being presented to medical staff, such a comparison result can be used as an indicator for, for example, changing the treatment policy.

Next, a second example of time scale alignment by the alignment function 113 will be described with reference to FIG. 4.

In the second alignment example, the treatment information 31 of the subject patient and the treatment information 33 of the similar-case patient are aligned with reference to each event 35. Specifically, in the example of FIG. 4, the processing circuitry 11 performs alignment, through the alignment function 113, by extending and contracting the times of the time scales of treatment information so that the vertical positions of each event 35 of the clinical pathways from “hospital admission” to “hospital discharge” of treatment information of both patients are aligned with each other. Even when there is an event that is not common to the clinical pathways, the time scales may be aligned as a whole by aligning the time scales so that common events are aligned with higher priority.

In the example of FIG. 4, it takes ten days for the subject patient from “surgery” to “thread removal”, whereas it takes about seven days for the similar-case patient from “surgery” to “thread removal”. Therefore, when the time scales are aligned with reference to each event 35, the graph. of the test information 34 of the similar-case patient is extended as indicated by the broken lines connecting dates in FIG. 4. Aligning time scales in this manner enables a comparison of the progress of vital sign information between events 35. It is possible to align the times scales of the treatment information 31 of the subject patient and the treatment information 33 of the similar-case patient with. respect to only events that the user wishes to compare, not, all events.

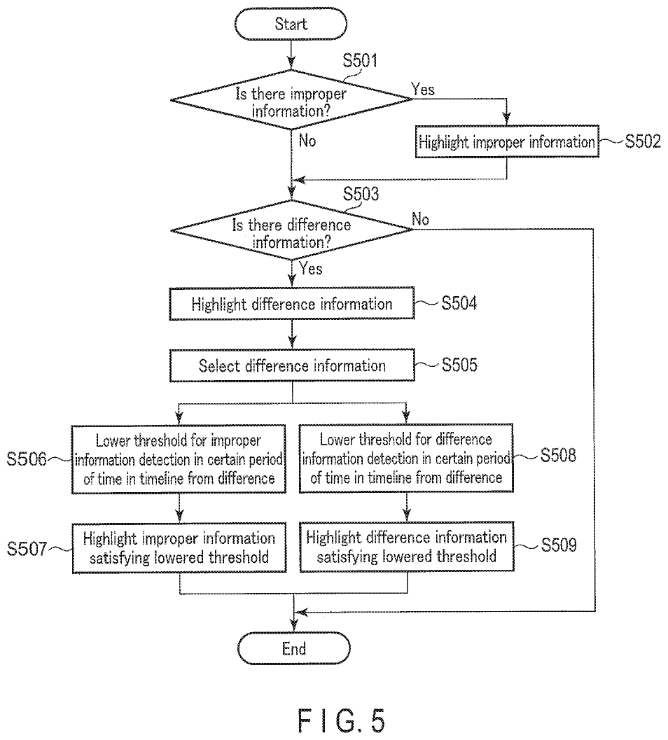

Next, details of the highlighting process in step S204 of FIG. 2 will be described with reference to the flowchart of FIG. 5.

In step S501, by executing the detection function 114, the processing circuitry 11 determines whether or not there is improper information. In the method for determining improper information, as stated above, an abnormal value or an outlier, which is a value deviated from the range determined as a normal range in general or from an average value by a threshold or more, in test information (such as vital signs) or results of tests (such as a blood test), is detected as improper information. In addition, when numerical values obtained by a test are graphed (subjected to function fitting) and when a slope of the graph is equal to or greater than a threshold, the relevant value may be determined as improper information. If there is improper information, the processing proceeds to step S502. If there is no improper information, the processing proceeds to step S503.

In step S502, by executing the display control function 115, the processing circuitry 11 highlights improper information. In the highlighting method, for example, the portion of improper information may be enclosed by a circle or polygon on the screen displayed on the GUI. Alternatively, improper information (an outlier or abnormal value) itself may be bolded, colored, or made to blink as long as improper information is displayed in a manner distinguishable from the other portions.

In step S503, by executing the detection function 114, the processing circuitry 11 determines through whether or not there is difference information between the subject patient information and the similar-case patient information. In the difference information determination method, for example, pattern matching may be performed on the test information of the subject patient and that of the similar-case patient and, if there is a portion (such as an item of medication information or image, or part of the vital sign waveform) where the difference is equal to or greater than a threshold, the portion may be detected as difference information. If there is difference information, the processing proceeds to step S504. If there is no difference information, the processing ends.

In step S504, by executing the display control function 115, the processing circuitry 11 highlights difference information. Any method similar to that of step S502 may be used as the highlighting method as long as difference information is displayed in a manner distinguishable from the other portions. All detected difference information items may be displayed on the GUI, or one difference information item with the greatest difference may be displayed on the GUI.

In step S505, by executing the display control function 115, the processing circuitry 11 selects difference information. Selection of difference information is assumed to be designated by the user, but difference information with the greatest difference may be automatically selected.

In step S506, by the display control function 115, the processing circuitry 11 lowers the threshold for detecting improper information within a certain period of time in the timeline with reference to the point of time of the difference information selected in step S505. The certain period of time may be, for example, a predetermined period of time on an hourly, daily, weekly, monthly or yearly basis, or may be a period of time between events, such as “surgery” and “hospital discharge”. The range of lowering the threshold is limited to the certain period of time because if the threshold is lowered in the entire range, a lot of improper information and difference information are detected, which may cause important information to be buried and thereby overlooked. Limiting the period of time to the period of time assumed to influence the selected difference information enables appropriate presentation of information.

How much the threshold is lowered varies depending on the type of information. For example, when the original threshold set for a vital sign as test information is “160 or higher systolic blood pressure”, the lowered threshold is, for example, “140 or higher systolic blood pressure”. When the original threshold set for medication information is “different drugs”, the lowered threshold may be “same drug with different dosages”.

Lowering the threshold may be executed for every information item within the certain period of time, or may be executed only for information related to the selected difference. For example, when blood pressure of vital signs is selected as difference information, a threshold for medication information on a blood pressure-lowering drug may be lowered with the thresholds for the other information, such as finding information, report information, and an image, unchanged.

In step S507, by executing the display control function 115, the processing circuitry 11 highlights improper information that is equal to or greater than the lowered threshold, if any.

In step S508, by executing the display control function 115, the processing circuitry 11 lowers the threshold for detecting difference information within a certain period of time in the timeline with reference to the point of time of the difference information selected in step S505. Lowering the threshold of difference information is the same process as that of step S506, and a description thereof is omitted.

In step S509, by executing the display control function 115, the processing circuitry 11 highlights difference information that is equal to or greater than the lowered threshold, if any.

Since the different portion and abnormal portion of a similar-case patient of a case similar to that of the subject patient are highlighted in steps S502 and S504, similar but different information can be prevented from being overlooked.

The processing circuitry 11 may detect, by executing the detection function 114, related information which is related to disease and injury information of interest of the subject patient from highlighted difference information. Related information includes, for example, vital sign information and medication information related to disease and injury information such as information on findings and disease name information. The related information may be associated with disease and injury information in a table or the like. Specifically, in the case where disease name information. “type 2 diabetes” is associated with medication information “hypoglycemic drug” in the table, when the disease and injury name of the subject patient is “type 2 diabetes”, medication information “hypoglycemic drug” is determined as related information.

The processing circuitry 11 highlights, by executing the display control function 115, at least the most related information of the related information. In the above example, at least difference information relating to a medication event may be further highlighted. The difference information relating to the related information may be further highlighted (by, for example, adopting two of the highlighting methods including bolding, coloring, and blinking), or the information other than the difference information relating to the related information may be non-highlighted.

Further, by lowering the threshold for, for example, a certain period of time including the point of time of difference information with reference to the point of time of the selected difference information in steps S508 and S509, improper information and difference information are further highlighted, which can assist detection of a cause and incident related to the selected difference information. Neither the operation of steps S506 and S507 nor the operation of steps S508 and S509 are given priority. Either operation may be executed earlier, or both operations may be executed at the same time. The highlighting processing is thereby finished.

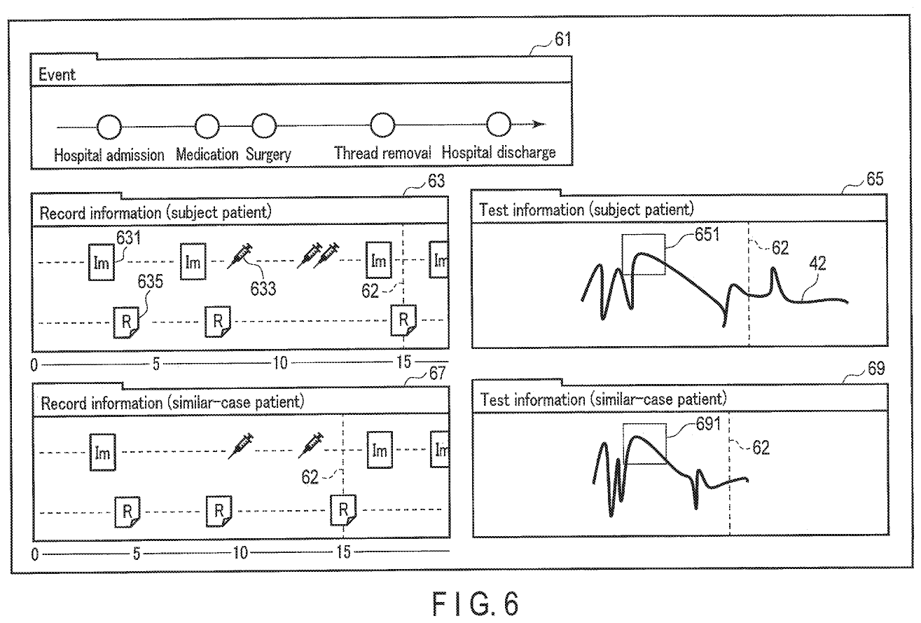

Next, a first example of highlighting according to the present embodiment will be described with reference to FIG. 6.

FIG. 6 is an example of a display screen of a GUI relating to an integrated display of medical information. The display screen is displayed on a display of, for example, a work station.

Event region 61, treatment record region 63 relating to treatment information of the subject patient, test information region 65 relating to test results of the subject patient, treatment record region 67 relating to treatment information of the similar-case patient, and test information region 69 relating to test results of the similar-case patient are displayed in parallel. Let us assume the case where the time scales of the displayed regions are aligned with reference to a certain event.

The event region 61 shows, for example, a clinical pathway. The event region 61 may be incorporated into the treatment record region or test information region. The treatment record regions 63 and 67 show information on medical examinations, such as a test and a treatment, with icons in time series. For example, icon 631 represents a medical image, icon 633 represents medication information, and icon 635 represents created report information. The test information regions 65 and 69 show vital signs, such as blood pressure.

The broken line 62 indicates a time common to the treatment record regions 63 and 67 and test information regions 65 and 69.

The event region 61, the treatment record region 63 of the patient, the test information region 65 of the patient, the treatment record region 67 of the similar case, and the test information region 69 of the similar case are displayed in separate windows, and the display positions may be configured to be switchable.

FIG. 6 is a display example of step S502, in which improper information in vital signs is highlighted, a rectangular highlight mark 651 is displayed on the relevant portion in an overlapping manner in test information region 65, and a rectangular highlight mark 691 is displayed on the relevant portion in an overlapping manner in test information region 69.

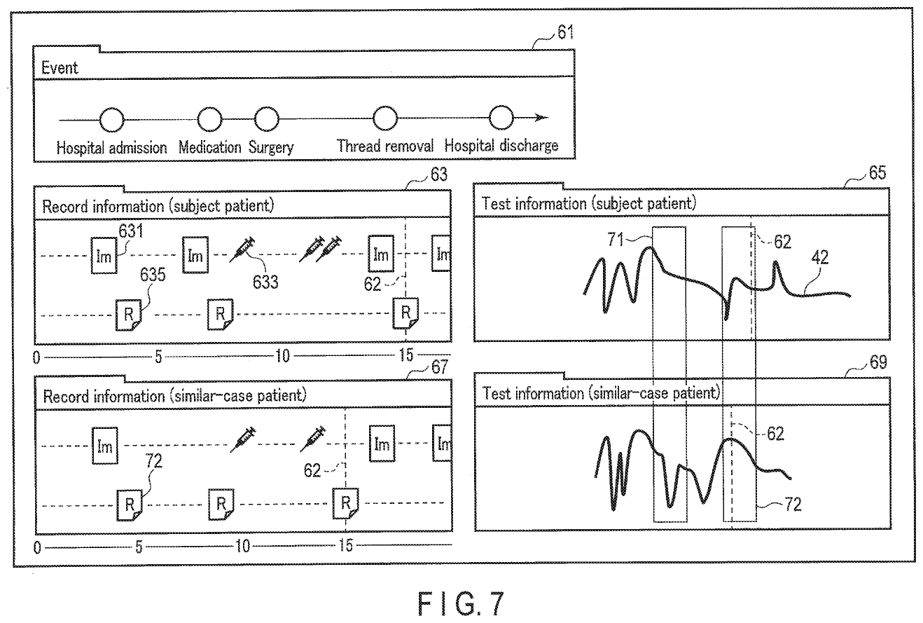

Next, a second example of highlighting according to the present embodiment will be described with reference to FIG. 7.

FIG. 7 is a display example of step S504, in which differences in vital sign information between the subject patient and the similar-case patient which are equal to or greater than a threshold are collectively highlighted by highlight marks 71 and 72 by means of the display control function 115. Highlight mark 71 may be separated between test information regions 65 and 69, and provided to each of the test information regions 65 and 69. Highlight mark 71 can highlight portions that are different between the subject patient and the similar-case patient. The portion highlighted by highlight mark 71 includes, for example, SOAP, anamnesis, a vital sign, a blood test result, and an image.

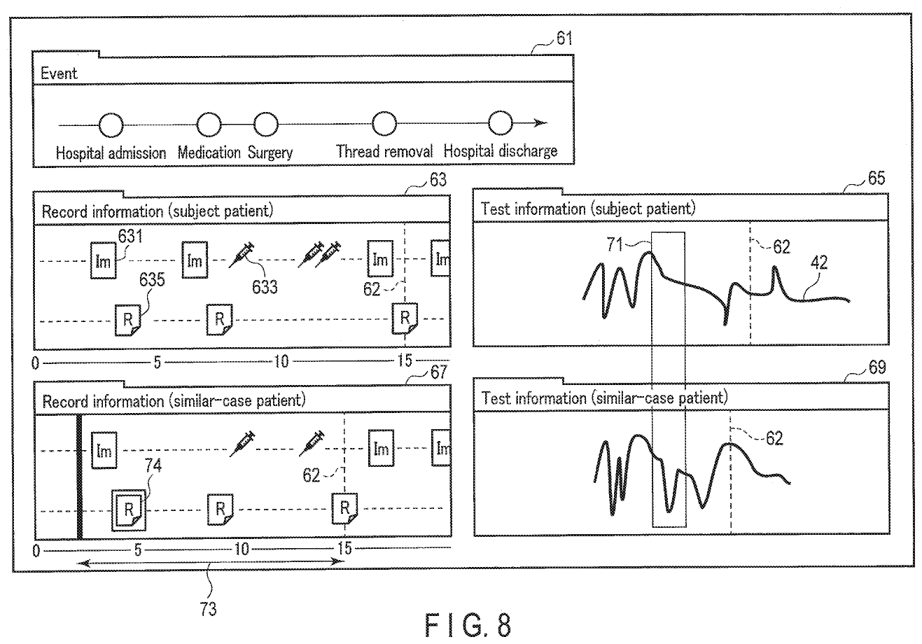

Next, a third example of highlighting according to the present embodiment will be described with reference to FIG. 8.

FIG. 8 is a display example of step S507, which shows a display state when a difference is selected by a user's designation from the display state of FIG. 7.

Let us assume that the difference indicated by highlight mark 71 is selected. The threshold for the “improper information” detection is lowered in a certain period of time 73 extending back from the point of time indicated by the highlight mark 71 along the timeline. Improper information not detected with the regular threshold is thereby provided with highlight mark 74. In the example of FIG. 8, improper information in the contents of the report is detected as a result of the lowering of the threshold, and is provided with highlight mark 74. This can increase the possibility of detecting events and indications causing the difference information provided with highlight mark 71. The certain period of time 73 may be a range extending back from the point of time indicated by the highlight mark 71 or a range extending forward from the point of time indicated by the highlight mark 71.

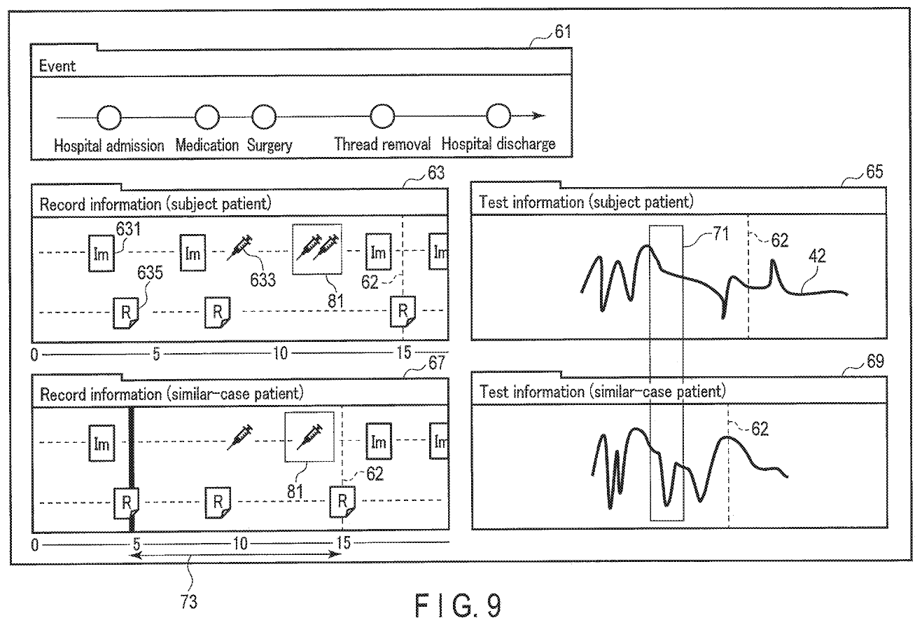

Next, a fourth example of highlighting according to the present embodiment will be described with reference to FIG. 9.

FIG. 9 is a display example of step S509, which shows a display state when a difference is selected by a user's designation from the display state of FIG. 7.

Highlight mark 71 is the same as that of FIG. 7; however, the threshold of “difference information” is lowered in the certain period of time 73. In the example of FIG. 9, when a difference in drug type is detected as difference information with the original threshold, a difference in dosage is detected as difference information with the lowered threshold even though the drug types are the same.

Accordingly, the difference in dosage of a drug, which is not detected with the regular threshold, is detected as a difference, and highlight mark 81 is provided to treatment record regions 63 and 67. Accordingly, as in the example of FIG. 8, the possibility of detecting events and indications causing the difference information provided with highlight mark 71 can be increased.

It is also possible to acquire information that although there is a difference, there is no difference in effect. For example, let us assume that highlight mark 81 of record information is selected by the user as difference information. When difference information is not detected from test information regions 65 and 69 even though the threshold is lowered, it can be determined that there is almost no difference in the changes in blood pressure due to the difference in drug (type/dosage).

According to the above-described embodiment, a similar-case patient of a case similar to the case of a subject patient is displayed in a manner enabling a comparison, and. improper information, which is an abnormal value and outlier, of the subject patient and the similar-case patient and difference information between the subject patient and the similar-case patient are highlighted. Accordingly, portions that are different between the similar-case patients and abnormal portions can be retrieved, and overlooking of information can be prevented.

When previous treatment information of the subject patient is used for comparison as similar-case patient information, the present treatment progress can be compared with the previous treatment progress, which can be used for consideration of a future treatment policy.

In addition, when difference information is detected, lowering the thresholds for improper information detection and difference information detection in a certain period of time from the point of time of the difference information assists detection of a cause that influences the difference information or a factor that the difference information may have influenced.

In addition, the functions of the embodiments may also be implemented by installing programs that execute respective processes in a computer, such as a work station, and loading them in the memory. The program that causes the computer to perform the technique can be stored and distributed by means of a storage medium, such as a magnetic disk (a hard disk, etc.), an optical disk (CD-ROM, DVD, Blu-ray (registered trademark) etc.), and a semiconductor memory.

While some embodiments have been described, the embodiments have been presented as examples, and are not intended to limit the scope of the invention. These embodiments can be implemented in various other forms, and various omissions, replacements, and changes can be made without departing from the spirit of the invention. The embodiments and their modifications are included in the scope and spirit of the invention and are included in the scope of the claimed inventions and their equivalents.

What is claimed is:

1. A medical information display apparatus comprising processing circuity configured to:
retrieve similar--case patient information based on subject patient information, the subject patient information including time-series treatment information on a subject patient, the similar-case patient information including time-series treatment information on a similar-case patient which has a case similar to a case of the subject patient;
detect, as difference information, information of a difference between the subject patient information and the similar-case patient information which is equal to or greater than a first threshold; and
cause the subject patient information and the similar-case patient information to be displayed and highlight the difference information.

2. The apparatus according to claim 1, wherein
the processing circuitry detects related information which is related to an injury or disease of interest from the highlighted difference information between the subject patient information. and the similar-case patient information, and further highlights at least most-related information from the related information.

3. The apparatus according to claim 1, wherein
the processing circuitry detects improper information in the subject patient information and the similar-case patient information, and highlights the improper information.

4. The apparatus according to claim 3, wherein
when the difference information is selected, the processing circuitry lowers a threshold for detecting the improper information in a first period including a point of time of occurrence of the difference information.

5. The apparatus according to claim 2, wherein
when the difference information is selected, the processing circuitry newly selects, as difference information, information of a difference equal to or greater than a second threshold which is lower than the first threshold in a first period including a point of time of occurrence of the difference information.

6. The apparatus according to claim 1, wherein
the similar-case patient information includes time-series treatment information on a previous treatment on the subject patient, and
the processing circuitry performs a retrieval in accordance with a designated retrieval condition to select a range of treatment information to be extracted as the similar-case patient information.

7. The apparatus according to claim 1, wherein
when the subject patient information and the similar-case patient information are displayed, the processing circuitry performs alignment so that time scales are aligned at least in part in the time series.

8. The apparatus according to claim 7, wherein
the processing circuitry performs alignment so that the time scales are aligned with reference to an event immediately before information of interest in the subject patient information of events.

9. The apparatus according to claim 7, wherein
the processing circuitry performs alignment so that the time scales of events are aligned with reference to each of the events.

10. The apparatus according to claim 1, wherein
the subject patient information includes a test result and treatment record of the subject patient,
the similar-case patient information includes a test result and treatment record of the similar-case patient, and
the processing circuitry causes the test result and treatment record of the subject patient and the test result and treatment record of the similar-case patient to be displayed in parallel in time series on a display screen.

11. The apparatus according to claim 3, wherein
the processing circuity causes the difference information and improper information displayed on a display screen to be displayed in a manner distinguishable from another region.

12. A medical information display system comprising a medical information display apparatus and an electric health record system, wherein
the medical information display apparatus comprises processing circuity configured to:
retrieve similar-case patient information based on subject patient information, the subject patient information including time-series treatment information on a subject patient, the similar-case patient information including time-series treatment information on a similar-case patient which has a case similar to a case of the subject patient;
detect difference information between the subject patient information and the similar-case patient information; and
cause the subject patient information and the similar-case patient information to be displayed and highlight at least difference information of a greatest difference of the difference information, and
the electronic health record system displays the subject patient information, the similar-case patient information, and the highlighted difference information.
